(19) United States
(12) Patent Application Publication
(10) Pub. No.: US 2003/0027337 A1
(43) Pub. Date: Feb. 6, 2003

(54) Sequence-specific DNA recombination in eukaryotic cells

(75) Inventors: Peter Droge, Koln (DE); Nicole Christ, Koln (DE); Elke Lorbach, Koln (DE)

Correspondence Address: Steven L. Highlander, FULBRIGHT & JAWORSKI L.L.P., Suite 2400, 600 Congress Avenue,, Austin, TX, 78701, US

(73) Assignee: Boehringer Ingelheim Pharma KG

(21) Appl. No.: 10/082,772

(22) Filed: Feb. 25, 2002

Related U.S. Application Data
(63) Continuation of application No. PCT/DE00/02947, filed on Aug. 29, 2000.

(30) Foreign Application Priority Data
Aug. 30, 1999 (DE) 199 41 186.7

(51) Int. Cl. C12N 15/86; C12N 5/00

(52) U.S. Cl. 435/456; 435/325

(57) ABSTRACT

The present invention relates to a method of sequence specific recombination of DNA in eukaryotic cells utilizing att sequences from the bacteriophage lambda. A particular embodiment of the invention relates to a method further comprising performing the sequence specific recombination of DNA with an Int and a Xis factor. The present invention further relates to vectors containing each of these sequences and their use as medicaments.

### BACKGROUND OF THE INVENTION

I. Field of the Invention

The present invention relates to a method of sequence specific recombination of DNA in eukaryotic cells, comprising the introducing of a first DNA sequence into a cell, introducing a second DNA sequence into a cell, and performing the sequence specific recombination by a bacteriophage lambda integrase Int. A preferred embodiment of the invention relates to a method, further comprising performing the sequence specific recombination of DNA by an Int and a Xis factor. The present invention further relates to vectors and their use as medicaments.

II. Description of Related Art

The controlled manipulation of eukaryotic genomes is an important method for investigation of the function(s) of specific genes in living organisms. Moreover, said manipulation plays a role in gene therapeutic methods in medicine. In this context the generation of transgenic animals, the change of genes or gene segments (so-called “gene targeting”) and the targeted integration for foreign DNA into the genome of higher eukaryotes are of particular importance. Recently these technologies could be improved by means of characterization and application of sequence specific recombination systems.

Conservative sequence specific DNA recombinases have been divided into two families. Members of the first family the so-called “integrase” family catalyze the cleavage and rejoining of DNA strands between two defined nucleotide sequences which will be named as recombination sequences in the following. The recombination sequences may be either on two different or on one and the same DNA molecule resulting in the inter- and the intramolecular recombination, respectively. In the latter case the result of the reaction depends on the respective orientation of the recombination sequences to each other. In the case of an inverted, i.e. opposite orientation of the recombination sequences an inversion of the DNA segments lying between the recombination sequences occurs. In the case of direct, i.e. tandem repeats of the recombination sequences on a DNA substrate a deletion occurs. In case of the intermolecular recombination, i.e. if both recombination sequences are located on two different DNA molecules a fusion of the two DNA molecules may occur. While members of the integrase family usually catalyze both intra- as well as intermolecular recombination the recombinases of the second family of the so-called “invertases/resolvases” are only able to catalyze the intramolecular recombination.

The recombinases which are used mainly for the manipulation of eukaryotic genomes at present belong to the integrase family. Said recombinases are the Cre recombinase of the bacteriophage P1 and the Flp recombinase from yeast (Müller, U. (1999) Mech. Develop., 82, pp. 3). The recombination sequences to which the Cre recombinase binds are named loxP. LoxP is a 34 bp long nucleotide sequence consisting of two 13 bp long inverted nucleotide sequences and an 8 bp long spacer lying between the inverted sequences (Hoess, R. et al. (1985) J. Mol. Biol., 181, pp. 351). The FRT named binding sequences for Flp are build up similarly. However, they differ from loxP (Kilby, J. et al. (1993) Trends Genet., 9, pp. 413). Therefor, the recombination sequences may not be replaced by each other, i.e. Cre is not able to recombine FRT sequences and FLP is not able to recombine loxP sequences. Both recombination systems are active over long distances, i.e. the DNA segment to be inverted or deleted and flanked by two loxp or FRT sequences may be several 10 000 base pairs long.

For example a tissue specific recombination in a mouse system, a chromosomal translocation in plants and animals and a controlled induction of the gene expression was achieved with said two systems; review article of Müller, U. (1999) Mech. Develop., 82, pp. 3. The DNA polymerase β was deleted in particular tissues of mice in this way; Gu, H. et al. (1994) Science, 265, pp. 103. A further example is the specific activation of the DNA tumor virus SV40 oncogene in the mouse lenses leading to tumor formation exclusively in these tissues. The Cre-loxP strategy was used beyond it also in connection with inducible promoters. For example the expression of the recombinase was regulated with an interferon-inducible promotor leading to the deletion of a specific gene in the liver and not— or only to a low extent—in other tissues; Kühn, R. et al. (1995) Science, 269, pp.1427.

So far two members of the invertase/resolvase family have been used for the manipulation of eukaryotic genomes. A mutant of the bacteriophage Mu invertase Gin can catalyze the inversion of a DNA fragment in plant protoplasts without cofactors. However, it has been discovered that this mutant is hyperrecombinative, i.e. it catalyzes DNA strand cleavages also at other than its naturally recombination sequences. This leads to unintended partially lethal recombination events in plant protoplast genomes. The β-recombinase from Streptococcus pyogenes catalyses the recombination in mouse cell cultures between two recombination sequences as directed repeats leading to the excision of the segment. However, simultaneously with deletion also inversion has been detected what renders the controlled use of the system for manipulation of eukaryotic genomes unsuitable.

The manipulation of eukaryotic genomes with the Cre and Flp recombinase, respectively, shows significant disadvantages. In case of deletion, i.e. the recombination of two tandem repeated loxP or FRT recombination sequences in a genome there is an irreversibly loss of the DNA segment lying between the tandem repeats. Thus, a gene located on this DNA segment will be lost permanently for the cell and the organism. Therefore, the reconstruction of the original state for a new analyses of the gene function e.g. in a later developmental stage of the organism is impossible. The irrevocable loss of the DNA segment caused by deletion may be avoided by an inversion of the respective DNA segment. A gene may be inactivated by an inversion without being lost and may be switched on again at a later developmental stage or in the adult animal by means of a timely regulated expression of the recombinase via back recombination. However, the use of both Cre and Flp recombinases in this modified method has the disadvantage that the inversion cannot be regulated as the recombination sequences will not be altered as a result of the recombination event. Thus, repeated recombination events occur causing the inactivation of the respective gene due to the inversion of the respective DNA segment only in some, at best in 50% of the target cells. There have been efforts to solve this problem at least in part by constructing mutated loxP sequences which cannot be used for further reaction after a single recombination. However, the disadvantage is the uniqueness of the reaction, i.e. there is no subsequent activation by back recombination after inactivation of the gene by inversion.

A further disadvantage of the Flp recombinase is its reduced heat stability at 37° C. limiting the efficiency of the recombination reaction in higher eukaryotes e.g. in mice having a body temperature of about 39° C. significantly. Therefor, Flp mutants have been constructed having a higher heat stability as the wild type recombinase, however, showing still a lower recombination efficiency than the Cre recombinase.

A use of sequence specific recombinases resides further in the medical field e.g. in gene therapy where the recombinases shall integrate a desired DNA segment into the genome of the respective human target cell in a stable and targeted way. Both Cre and Flp may catalyze intermolecular recombination. Both recombinases recombine a plasmid DNA which carries a copy of its respective recombination sequence with a corresponding recombination sequence which has been inserted into the eukaryotic genome via homologous recombination before. However, it is desirable that this reaction is feasible with a “naturally” occurring recombination sequence in the eukaryotic genome. As loxP and FRT are 34 and 54 nucleotides long, respectively, an occurrence of this recombination sequences as part of the genome is statistically extreme unlikely. Even if a recombination sequence is present the disadvantage of the afore described back reaction still exists, i.e. both Cre and Flp recombinases may excise the inserted DNA segment after successful integration by intramolecular recombination.

Thus, one problem of the present invention is to provide a simple and regulatable recombination system and the required working means. A further problem of the present invention is the provision of a recombination system and the required working means, which may carry out a stable and targeted integration of a desired DNA sequence.

Said problems are solved by the subject matter characterized in the claims. The invention is explained in more detail with the following illustrations.

### SUMMARY OF THE INVENTION

The term “transformation” or “to transform” as used herein means any introducing of a nucleic acid sequence into a cell. The introduction may be e.g. a transfection or lipofection or may be carried out by means of the calcium method, electroshock method or an oocyte injection. The term “transformation” or “to transform” also means the introduction of a viral nucleic acid sequence comprising e.g. the recombination sequence(s) and a therapeutic gene or gene fragment in a way which is for the respective virus the naturally one. The viral nucleic acid sequence needs not to be present as a naked nucleic acid sequence but may be packaged in a viral protein envelope. Thus, the term relates not only to the method which is usually known under the term “transformation” or “to transform”.

The term “derivative” as used herein relates to attB and attP sequences and attL and attR sequences having modifications in the form of one or more, at most six, preferably two, three, four or five substitutions in contrast to naturally occurring recombination sequences.

The term “homologue” or “homologous” or “similar” as used herein with regard to recombination sequences relates to a nucleic acid sequence being identical for about 70%, preferably for about 80%, more preferably for about 85%, further more preferably for about 90%, further more preferably for about 95%, and most preferably for about 99% to naturally occurring recombination sequences.

The term “vector” as used herein relates to naturally occurring or synthetically generated constructs for uptake, proliferation, expression or transmission of nucleic acids e.g. plasmids, phagemids, cosmids, artificial chromosomes, bacteriophages, viruses or retro viruses.

The integrase (usually and designated herein as “Int”) of the bacteriophage lambda belongs like Cre and Flp to the integrase family of the sequence specific conservative DNA recombinases. Int catalyses the integrative recombination between two different recombination sequences namely attB and attP. AttB comprises 21 nucleotides and was originally isolated from the E. coli genome; Mizuuchi, M. and Mizuuchi, K. (1980) Proc. Natl. Acad. Sci. USA, 77, pp. 3220. On the other hand attP having 243 nucleotides is much longer and occures naturally in the genome of the bacteriophage lambda; Landy, A. and Ross, W. (1977) Science, 197, pp. 1147. The Int recombinase consists of seven binding sites altogether in attP and two in attB. The biological function of Int is the sequence specific integration of the circular phage genome into the locus attB on the E. coli chromosome. Int needs a protein co-factor the so-called integration host factor (usually and designated herein as “IHF”) for the integrative recombination; Kikuchi, Y. und Nash, H. (1978) J. Biol. Chem., 253, 7149. IHF is needed for the assembly of a functional recombination complex with attP. A second co-factor for the integration reaction is the DNA negative supercoiling of attP. Finally, the recombination between attB and attP leads to the formation of two new recombination sequences, namely attL and attR, which serve as substrate and recognition sequence for a further recombination reaction, the excision reaction. A comprehensive summary of the bacteriophage lambda integration is given e.g. in Landy, A. (1989) Annu. Rev. Biochem., 58, pp. 913.

The excision of the phage genome out of the bacterial genome is catalyzed by the Int recombinase also. For this, a further co-factor is needed in addition to Int and IHF, which is encoded from the bacteriophage lambda also. This is the excisionase (usually and designated herein as “XIS”) having two binding sites in attR; Gottesman, M. and Weisberg, R. (1971) The Bacteriophage Lambda, Cold Spring Harbor Laboratory, pp.113. In contrast to the integrative recombination DNA negative supercoiling of the recombination sequences is not necessary for the excisive recombination. However, DNA negative supercoiling increases the efficiency of the recombination reaction. A further improvement of the efficiency of the excision reaction may be achieved with a second co-factor namely FIS (factor for inversion stimulation) which acts in connection with Xis; Landy, A. (1989) Annu. Rev. Biochem., 58, pp.913. The excision is genetically the exact reverse reaction of the integration, i.e. attB and attP are generated again. A comprehensive summary of the bacteriophage lambda excision is given e.g. in Landy, A. (1989) Annu. Rev. Biochem., 58, pp. 913.

One aspect of the present invention relates to a method of sequence specific recombination of DNA in eukaryotic cells, comprising a) the introduction of a first DNA sequence into a cell, b) the introduction of a second DNA sequence into a cell, and c) performing the sequence specific recombination by a bacteriophage lambda integrase Int. Preferred is a method wherein the first DNA sequence comprises an attB sequence according to SEQ ID NO:1 or a derivative thereof and the second DNA sequence comprises an attP sequence according to SEQ ID NO:2 or a derivative thereof. Further preferred is a method wherein the first DNA sequence comprises an attL sequence according to SEQ ID NO:3 or a derivative thereof and the second DNA sequence comprises an attR sequence according to SEQ ID NO:4 or a derivative thereof, wherein in step c) the sequence specific recombination is performed by an Int and a Xis factor.

The method of the present invention may be carried out not only with the naturally occuring attB and/or attP sequences or the attL and/or attR sequences but also with modified e.g. substituted attB and/or attP sequences or the attL and/or attR sequences. For example an integrative recombination of the bacteriophage lambda and E. coli between attP and attB homologous sequences (mutants of the wild-type sequences) have been observed which have one or a combination of the following substitutions at the following positions in attB: G, T (at position −9); A, C, G (−8); C, A, T (−7); T, G, A (−6); C, A (−5); A (−4); G, A (−3); A, C, G (−2); A, C, G (−1); A, C, G (0); T, C, G (+1); A, C, G (+2); T, G, C (+3); A, G, T (+4); A, C, G (+5); G, T (+6); G, T (+7); G, T, A (+8); C, G, A (+9); C, G, A (+10); T, A, C (+11) (Nash, H. (1981) Annu. Rev. Genet., 15, pp. 143; Nussinov, R. and Weisberg, R. (1986) J. Biomol. Struct. Dynamics, 3, pp 1134) and/or in attP: T (at position +1); C (+2) and A (+4); Nash, H. (1981) Annu. Rev. Genet., 15, pp.143.

Thus, the present invention relates to a method wherein the used attB and attP sequences have one or more substitutions in comparison to the naturally occuring attB sequence according to SEQ ID NO:1 and the attP sequence according to SEQ ID NO:2, respectively. Furthermore, the present invention relates to a method wherein the used attL and attR sequences have one or more substitutions in comparison to the naturally occuring attL sequence according to SEQ ID NO:3 and the attR sequence according to SEQ ID NO:4, respectively. Preferred is a method wherein the recombination sequences have one, two, three, four or five substitutions. The substitutions may occur both in the overlap region (see FIG. 6A, open rectangle) and in the core region (see FIG. 6A, dash). The complete overlap region comprising seven nucleotides may be substituted also. More preferred is a method wherein substitutions are introduced into the attB and attP sequence either in the core region or in the overlap region. Preferred is the introduction of a substitution in the overlap region and the simultaneous introduction of one or two substitutions in the core region.

For the method of the present invention it is not necessary to introduce a corresponding substitution in attP if a substitution in attB is introduced or to introduce a corresponding substitution in attR if a substitution in attL is introduced and vice versa. A modification in the form of a substitution into recombination sequences is to be chosen such that the recombination can be carried out in spite of the modification(s). Examples for such substitutions are listed e.g. in the publications of Nash, H. (1981), supra and Nussinov, R. and Weisberg, R. (1986), supra and are not considered to be limiting. Further modifications may be easily introduced e.g. by mutagenesis methods and may be tested for their use by test recombinations.

Thus, the present invention relates further to a method wherein either the used attB sequence in comparison to the naturally occurring attB sequence according to SEQ ID NO:1 or the used attP sequence in comparison to the naturally occurring attP sequence according to SEQ ID NO:2, or either the used attL sequence in comparison to the naturally occurring attL sequence according to SEQ ID NO:3 or the used attR sequence in comparison to the naturally occurring attR sequence according to SEQ ID NO:4 have one or more substitutions. Therefore, one or more substitutions in one of the recombination sequences does not necessarily imply to the corresponding substitution in the other recombination sequence.

In a preferred embodiment of the method of the present invention the attB sequence comprise 21 nucleotides and corresponds to the originally isolated sequence from the E. coli genome (Mizuuchi, M. and Mizuuchi, K. (1980) Proc. Natl. Acad. Sci. USA, 77, pp. 3220) and the attP sequence comprises 243 nucleotides and corresponds to the originally isolated sequence from the bacteriophage lambda genome; Landy, A. and Ross, W. (1977) Science, 197, pp. 1147.

In a further preferred embodiment of the method of the present invention the attL sequence comprises 102 nucleotides and the attR sequence comprises 162 nucleotides both sequences corresping to the originally isolated sequences from the E. coli genome; Landy, A. (1989) Annu. Rev. Biochem., 58, pp.913.

In order to perform the method of the present invention in addition to the recombination sequence the first DNA sequence may comprise further DNA sequences which allow the integration into a desired target locus in the genome of the eukaryotic cell. This recombination occurs via the homologous recombination which is mediated by internal cellular recombination mechanisms. For said recombination the further DNA sequences have to be homologous to the DNA of the target locus and located as well as 3′ and 5′ of the attB and attL sequences, respectively. The person skilled in the art knows how great the degree of the homology and how long the respective 3′ and 5′ sequences have to be such that the homologous recombination occurs with a sufficient probability; see review of Capecchi, M. (1989) Science, 244, pp. 1288.

The second DNA sequence with the attP and attR recombination sequences, respectively, may also comprise DNA sequences which are necessary for an integration into a desired target locus via homologous recombination. For the method of the present invention as well as the first and/or the second DNA sequence may comprise the further DNA sequences. Preferred is a method wherein both DNA sequences comprise the further DNA sequences.

The introduction of the first and second DNA sequence with or without further DNA sequences may be performed both consecutively and in a co-transformation wherein the DNA sequences are present on two different DNA molecules. Preferred is a method, wherein the first and second DNA sequence with or without further DNA sequences are present and introduced into the eukaryotic cells on a single DNA molecule. Furthermore, the first DNA sequence may be introduced into a cell and the second DNA sequence may be introduced into another cell wherein the cells are fused subsequently. The term fusion means crossing of organisms as well as cell fusion in the widest sense.

The method of the present invention may be used e.g. to invert the DNA segment lying between the indirectly orientated recombination sequences in a intramolecular recombination. Furthermore, the method of the present invention may be used to delete the DNA segment lying between the directly orientated recombination sequences in a intramolecular recombination. If the recombination sequences are each incorporated in 5′-3′ or in 3′-5′ orientation they are present in direct orientation. The recombination sequences are in indirect orientation if e.g. the attB sequence is integrated in 5′-3′ and the attP sequence is integrated in 3′-5′ orientation. If the recombination sequences are each incorporated via homologous recombination in intron sequences 5′ and 3′ of an exon and the recombination is performed by an integrase the exon would be inverted in case of indirectly orientated recombination sequences and deleted in case of directly orientated recombination sequences, respectively. With this procedure the polypeptide encoded by the respective gene may lose its activity or function or the transcription may be stopped by the inversion or deletion such that no (complete) transcript is generated. In this way e.g. the biological function of the encoded polypeptide may be investigated.

However, the first and/or second DNA sequence may comprise further nucleic acid sequences encoding one or more polypeptides of interest. For example a structural protein, an enzyme or a regulatory protein may be introduced via the recombination sequences into the genome being transiently expressed after intramolecular recombination. The introduced polypeptide may be an endogenous or exogenous one. Furthermore, a marker protein may be introduced. The person skilled in the art knows that this listing of applications of the method according to the present invention is only exemplary and not limiting. Examples of applications according to the present invention performed with the so far used Cre and Flp recombinases may be found e.g. in the review of Kilby, N. et al., (1993), Trends Genet., 9, pp.413.

Furthermore, the method of the present invention may be used to delete or invert DNA segments on vectors by an intramolecular recombination on episomal substrates. A deletion reaction may be used e.g. to delete packaging sequences from so-called helper viruses. This method has a broad application in the industrial production of viral vectors for gene therapeutic applications; Hardy, S. et al., (1997), J. Virol., 71, pp.1842.

The intermolecular recombination leads to the fusion of two DNA molecules each having a copy of attB and attP or attL and attR. For example, attB may be introduced first via homologous recombination in a known well characterized genomic locus of a cell. Subsequently an attP carrying vector may be integrated into said genomic attB sequence via intermolecular recombination. Preferred in this method is the expression of the mutant integrase Int-h/218 the gene of which is located on a second DNA vector being co-transfected. Further sequences may be located on the attP carrying vector, e.g. a gene for a particular marker protein flanked by loxP/FRT sequences. With this approach it may be achieved that e.g. in comparative expression analyses of different genes in a cell type said genes are not influenced by positive or negative influences of the respective genomic integration locus.

To perform the method of the present invention an integrase has to act on the recombination sequences. The integrase or the integrase gene and/or the Xis factor or the Xis factor gene may be present in the eukaryotic cell already before introducing the first and second DNA sequence. They may also be introduced between the introduction of the first and second DNA sequence or after the introduction of the first and second DNA sequence. The integrase used for the sequence specific recombination is preferably expressed in the cell in which the reaction is carried out. For that purpose a third DNA sequence comprising an integrase gene is introduced into the cells. If the sequence specific recombination is carried out with attL/attR a Xis factor gene (fourth DNA sequence) may be introduced into the cells in addition. Most preferred is a method wherein the third and/or fourth DNA sequence is integrated into the eukaryotic genome of the cell via homologous recombination or randomly. Further preferred is a method wherein the third and/or fourth DNA sequence comprise regulatory sequences resulting in a spatial and/or temporal expression of the integrase gene and/or Xis factor gene.

In this case a spatial expression means that the recombinase and the Xis factor, respectively, is expressed only in a particular cell type by use of cell type specific promotors and catalyses the recombination only in these cells, e.g. in liver cells, kidney cells, nerve cells or cells of the immune system. In the regulation of the integrase/Xis factor expression a temporal expression may be achieved by means of promoters being active from or in a particular developmental stage or at a particular point of time in an adult organism. Furthermore, the temporal expression may be achieved by use of inducible promoters, e.g. by interferon or tetracycline depended promotors; see review of Müller, U. (1999) Mech. Develop.,82, pp. 3.

The integrase used in the method of the present invention may be both the wild-type and the modified integrase of the bacteriophage lambda. As the wild-type integrase is only able to perform the recombination reaction with a co-factor, namely IHF, it is preferred to use a modified integrase in the method of the present invention. If the wild-type integrase is used in the method of the present invention IHF is needed for the recombination reaction in addition. The modified integrase is modified such that said integrase may carry out the recombination reaction without IHF. The generation of modified polypeptides and screening for the desired activity is state of the art and may be performed easily; Erlich, H. (1989) PCR Technology. Stockton Press. Two Int mutants are preferred bacteriophage lambda integrases designated as Int-h and Int-h/218; Miller et al. (1980) Cell, 20, pp. 721; Christ, N. and Dröge, P. (1999) J. Mol. Biol., 288, pp. 825. Int-h includes a lysine residue instead of a glutamate residue at position 174 in comparison to wild-type Int. Int-h/218 includes a further lysine residue instead of a glutamate residue at position 218 and was generated by PCR mutagenesis of the Int-h gene. Said mutants may catalyze as well as the recombination between attB/attP and also between attL/attR without the co-factors IHF, Xis and negative super-coiling in E. coli and in vitro, i.e. with purified substrates in a reaction tube. In eukaryotic cells the mutants need only the co-factor Xis for the recombination between attL/attR. A further improvement of the efficiency of the recombination between attL/attR may be achieved with a further co-factor, e.g. FIS. The mutant Int-h/218 is preferred, because this mutant may catalyze the co-factor independent integrative reaction with increased efficiency; Christ, N. and Dröge, P. (1999) J. Mol. Biol., 288, pp. 825.

The method of the present invention may be performed in all eukaryotic cells. The cells may be present e.g. in a cell culture and comprise all types of plant and animal cells. For example the cells may be oocytes, embryonic stem cells, hematopoietic stem cells or any type of differentiated cells. A method is preferred wherein the eukaryotic cell is a mammalian cell. More preferred is a method wherein the mammalian cell is a human, simian, murine, rat, rabbit, hamster, goat, bovine, sheep or pig cell.

Furthermore, a preferred embodiment of the present invention relates to a method wherein optionally a second sequence specific recombination of DNA is performed by a bacteriophage lambda integrase and a Xis factor. The second recombination needs the attL and attR sequences generated by a first recombination of attB and attP or the derivatives thereof. Therefore, the second sequence specific recombination is restricted to a method using in the first sequence specific recombination the attB and attP sequences or the derivatives thereof. Both wild-type and Int mutants can only catalyze the so-called integrative recombination without addition of further factors, i.e. they recombine attB with attP and not attL with attR if stably integrated into the genome of the cells. The wild-type integrase needs for the so-called excision recombination the factors IHF, Xis and negative super coiling. The Int mutants Int-h and Int-h/218 need for the excision recombination only the Xis factor. Thus, it is possible to run off two recombination reactions one after the other in a controlled manner if further factors for the second recombination reaction namely the excision reaction are present in the cell. Together with other already used recombination systems new strategies may be developed for the controlled manipulation of higher eukaryotic genomes. This is possible because the different recombination systems use only their own recombination sequences.

For example the Int system may be used to integrate loxP and/or FRT sequences in a targeted way into a genomic locus of a eukaryotic genome and to activate and inactivate, respectively, a gene subsequently by controlled expression of Cre and/or Flp. The Int system may be used further to delete loxP/FRT sequences from the genome after use, i.e. the recombination with the respective recombinase.

Furthermore, a method is preferred wherein a further DNA sequence comprising a Xis factor gene is introduced into the cells. Most preferred is a method wherein the further DNA sequence further comprises a regulatory DNA sequence giving rise to a spatial and/or temporal expression of the Xis factor gene.

For example, after successful integrative intramolecular recombination (inversion) by means of Int leading to the activation/inactivation of a gene in a particular cell type said gene may be inactivated or activated at a later point of time again by means of the induced spatial and/or temporal expression of Xis with the simultaneously expression of Int.

Furthermore, the invention relates to the use of an attB sequence according to SEQ ID NO:1 or the derivative thereof and to an attP sequence according to SEQ ID NO:2 or the derivative thereof, or an attL sequence according to SEQ ID NO:3 or the derivative thereof and to an attR sequence according to SEQ ID NO:4 or the derivative thereof, in a sequence specific recombination of DNA in eukaryotic cells. The eukaryotic cell may be present in a cell aggregate of an organism, e.g. a mammal, having no integrase or Xis factor in its cells. Said organism may be used for breeding with other organisms having in their cells the integrase or the Xis factor so that offsprings are generated wherein the sequence specific recombination is performed in cells of said offsprings. Thus, the invention relates also to the use of an integrase or an integrase gene and a Xis factor or a Xis factor gene in a sequence specific recombination in eukaryotic cells.

The inventors have identified a sequence in the human genome (designated herein as attH) having a homology of about 85% to attB. AttH may be used as a recombination sequence for the integration of foreign DNA into the human genome. Therefor, the second recombination sequence attP may be modified accordingly so that the integrase can perform the recombination reaction with high efficiency. The inventors could demonstrate that attH can be recombined with a version of attP modified by the inventors which is designated herein as attp* and depicted as SEQ ID NO:5 by means of Int-h in E. coli. Experiments with human cells demonstrated that attH is recombined with attP* also as part of the human genome if Int-h is transiently synthesized by said cells.

The possibility follows that a foreign circular DNA having an attP recombination sequence may be stably integrated into the naturally occurring attH locus of the human genome in a targeted way. AttH is only one example for a recombination sequence naturally occurring in the human genome. Further sequences may be identified within the Human Genome Project having a homology to attB and may be used for the integration of a foreign DNA into the human genome also. Dependent on said sequence present in the human genome and being homologous to attB a corresponding attP recombination sequence in the foreign circular DNA is chosen. Preferred is a foreign circular DNA including the nucleic acid sequence of the naturally occurring attP sequence. More preferred is a derivative of the naturally occurring attP sequence having at most six, preferably one to five, in particular three substitutions. Most preferred is a foreign circular DNA comprising the attP* nucleic acid sequence according to SEQ ID NO:5 having a homology of about 95% to attP.

The integrase may be delivered into the cells either as a polypeptide or via an expression vector. The integrase gene may be present, furthermore, as an expressable nucleic acid sequence on the DNA molecule which comprises the modified or naturally attP sequence or the attP* sequence.

The foreign circular DNA including the natural attP sequence or the derivative or homologue thereof, in particular the attP* sequence according to SEQ ID NO:5, comprises also the therapeutic gene or gene fragment to be introduced into the genome. Therapeutic genes may be e.g. the CFTR gene, the ADA gene, the LDL receptor gene, the β-globin gene, the Factor VIII or Factor IX gene, the alpha-1-antitrypsin gene or the dystropin gene. The foreign circular DNA may be e.g. a viral vector already used in somatic gene therapies. The vector may be also cell specific so that it only transfects those cells which are desired for the gene therapy e.g. epithelial lung cells, bone marrow stem cells, T lymphocytes, B lymphocytes, liver cells, kidney cells, nerve cells, skeletal muscle cells, hematopoietic stem cells or fibroblasts. The person skilled in the art knows that this listing is only a selection of therapeutic genes and target cells and other genes and target cells may be used for gene therapy also. Gen fragments comprise e.g. deletions of therapeutic genes, single exons, antisense nucleic acid sequences or ribozymes. Furthennore, gene fragments may comprise segments of a gene including trinucleotide repeats of a gene e.g. the fragile-X-syndrome gene.

IHF must be present if the wild-type integrase is used in a recombination. Preferred is the use of a modified integrase wherein the recombination may occur without IHF. Particularly preferred is the use of Int-h or Int-h/218.

Thus, the present invention relates to the naturally occurring attP sequence or the derivative or homologue thereof. Particularly the invention relates to the attP* nucleic acid sequence according to SEQ ID NO:5. Furthermore, the present invention relates to a vector comprising the naturally occurring attP sequence or the derivative thereof, particularly the attP* nucleic acid sequence according to SEQ ID NO:5 and a further nucleic acid sequence comprising a therapeutic gene or the gene fragment thereof Preferred is a vector wherein the therapeutic gene comprises a CFTR gene, ADA gene, LDL receptor gene, alpha or beta globin gene, alpha-1-antitrypsin gene, Factor VIII or Factor IX gene or the fragment thereof. The vector may comprise regulatory DNA elements, too, regulating the expression of the therapeutic gene or the gene fragment thereof.

Furthermore, the present invention relates to the use of the vector as a medicament for human or veterinary medicine. Further, the invention relates to the use of the vector for the manufacture of a medicament for the somatic gene therapy.

The vectors of the present invention may be administered e.g. by intravenous or intramuscular injections. The vectors may be also taken up by aerosols. Further applications are obvious for the person skilled in the art.

### BRIEF DESCRIPTION OF THE DRAWINGS

FIG. 1 shows a schematic presentation of the recombination reactions namely integration and excision catalyzed by the integrase Int. A super helical plasmid DNA (top) carrying a copy of the recombination sequence attP is shown. AttP consists of five so-called arm binding sites for Int (P1, P2, P1′, P2′, P3′), two core Int binding sites (C and C′; marked with black arrows), three binding sites for IHF (H1, H2, H′), two binding sites for Xis (X1, X2) and the so-called overlap region (open rectangle) where the actual DNA strand exchange takes place. The partner sequence for attP, attB, is shown on an linear DNA segment beneath and consists of two core binding sites for Int (B and B′; marked with open arrows) and the overlap region. For the recombination between attB and attP Int and IHF are necessary, leading to the integration of the plasmid into the DNA segment carrying attB. Thereby, two new hybrid recombination sequences attL and attR are formed serving as target sequences for the excision. This reaction requires Int and IHF and a further cofactor Xis encoded by the phage lamda.

FIG. 2A shows a schematic presentation of the integrase expression vectors and FIG. 2B shows a schematic presentation of a Western analysis. (FIG. 2A): The vector pKEXInt includes the wild-type integrase gene, the vector pKEXInt-h includes the gene of the mutant Int-h and the vector pKEXInt-h/218 includes the gene of the mutant Int-h/218. The control vector (pKEX) includes no Int gene. The respective genes for the wild-type integrase (Int) and the two mutants (Int-h and Int-h/218) are shown as gray bars. Following the coding regions signal sequences for RNA processing are present which should guaranty an increased intracellular stability of the respective mRNA (dotted rectangles) and are designated as SV40, t-Ag splice and polyA signals. The expression of the integrase genes occurs through the human cytomegalo virus (CMV) promotor. (FIG. 2B): After introducing the respective vector, as shown, into the reporter cell lines B2 and B3 cell lysates were prepared and proteins were separated upon their molecular weight in a SDS page. The presence of the Int-h protein was made visible through polyclonal mouse antibodies against wild-type Int (lanes 2 and 4). The position of Int in the gel is marked with an arrow.

FIG. 3 shows a schematic presentation of the substrate vectors. (A): pGFPattB/attP. Depicted is the vector linearized with ApaLI. The big black arrows mark the position and orientation of the two recombination sequences attB and attP which flank the GFP (green fluorescent protein) gene, which in turn is placed in inverted orientation to the CMV promotor. PA designates the polyA signal. The neo resistance gene which is expressed by the SV40 promotor and enables the selection of stable reporter cell lines is additionally lying on the vector. Recognition sites for the restriction enzyme NcoI are marked also. The integrative recombination between attB and attP leads to the inversion of the GFP gene and, thus, to its expression. The small open and closed arrows mark the position and orientation of the single PCR primers and are designated as p1 to p7. (B): pGFPattL/attR. The vector is identical to pGFPattB/attP, however, includes attL and attR instead of attB and attP. The GFP gene is lying in 3′-5′ orientation to the CMV promotor. The hatched box designates the position of the probe which was used for the Southern analysis.

FIGS. 4A-4D show schematically the detection of the integrative recombination in reporter cell lines by means of PCR after separation of the DNA molecules in agarose gels (1.2% w/v) in which DNA was made visible by staining with ethidium bromide. (FIG. 4A): Reverse transcriptase PCR (RT-PCR). The vectors pKEX and pKEXInt-h (FIG. 2A) were separately introduced into the respective reporter cell line B1 to B3 by electroporation. Proceeding from isolated polyA mRNA the RT-PCR analysis shows the expected product with the primer pair p3/p4 (FIG. 3) only if the cells were treated with pKEXInt-h (lanes 1, 3 and 5). The β-actin gene from the same RNA preparations was amplified as a control of the RNA content. Lane M: DNA ladder; lane O: RT-PCR control without RNA template. (FIGS. 4B and 4C): Genomic PCR analysis. From the respective cell lines genomic DNA was isolated 72 hours after electroporation and amplified with the primer pairs p3/p4 (FIG. 3) and p1/p2 (FIG. 3). The numbering and designation of the lanes correspond to FIG. 4A. (FIG. 4D): Deletion test. Isolated genomic DNA was amplified with the primer pair p5/p6 (FIG. 3). The position of the PCR product (420 bp) which is expected after deletion instead of inversion is marked with an arrow. The numbering and designation of the lanes correspond to FIG. 4A.

FIGS. 5A and 5B show schematically the detection of the inversion in reporter cell lines by PCR and Southern hybridization after separation of the DNA molecules in agarose gels (1.2% w/v). (FIG. 5A): PCR analysis. A fraction of genomic DNA which was isolated from cell lines B1, B2, B3 and BL60 which were treated with vectors pKEX and pKEXInt-h was amplified with primer pairs p3/p4 and p5/p7 (FIG. 3). The PCR products going back to the inversion of the GFP gene catalysed by the integrase are visible in lanes 1, 3 and 5. Lane M: DNA ladder; lane O: PCR control without genomic DNA. (FIG. 5B) Southern analysis: The rest of the fraction of the analyzed DNA shown in FIG. 5A was incubated with the restriction enzyme NcoI separated in an agarose gel electrophoresis upon its molecular weight and transferred on a nitrocellulose membrane subsequently. GFP carrying DNA fragments were made visible by means of a radioactive labeled probe (FIG. 3B) to detect the recombination. Lane 9: unrecombined pGFPattB/attP; lane 10: recombined pGFPattB/attP.

FIG. 6A shows a presentation of nucleic acid sequences comprising attB and attH, respectively. FIG. 6B shows a representation of partial sequences of attP and attP*. (A): Sequence comparison between attB and attH. The Int core binding sites B and B′ in attB are marked with a dash in top of the sequences. The Int core binding sites H and H′ in attH are marked with a dashed line in top of the sequences. The overlap sequences are characterized by open rectangles. Differences in the sequences are marked with a perpendicular double dashes. The numbering of the residues in the core and overlap regions relate to the center of the overlap designated with O and defined by Landy and Ross ((1977), Science, 197, pp.1147). The sequence from −9 to +11 is the attB and attH site, respectively. (B): Sequence comparison between the partial sequences of attP and attP*, corresponding to attB and attH, respectively. The designations are used as in FIG. 6A.

FIG. 7 shows schematically the detection of the recombination between attH and attP* on the vector pACH in E. coli after separation in an agarose gel electrophoresis. The substrate vector pACH was co-transformed together with the respective prokaryotic expression vectors for Int, Int-h or Int-h/218 into E. coli strain CSH26 or CSH26 delta IHF. Plasmid DNA was isolated 36 hours after selection, incubated with the restriction enzymes HindIII and AvaI, separated and made visible by agarose gel electroporesis. The position of the restriction fragments generated by inversion are marked as “invers.” The position of the DNA which has not recombined is marked as pACH. Lanes 1 and 12: DNA ladder; Lanes 2 and 3: expression vector and DNA of non recombined pACH; Lanes 4 to 7: DNA isolated from CSH26; Lanes 8 to 11: DNA isolated from CSH delta IHF.

FIG. 8 shows schematically the strategy for the integration of the vector pEL13 into the genomic locus attH and the principle of the detection method. The integration vector pEL13 carries a resistance gene (arrow marked with “hygr”), the gene for Int-h (arrow marked with “int h”) under the control of the CMV promoter and a copy of attP* (open rectangle marked with “att P*/P*OP*′”). Int-h is expressed after introducing of the vector into BL60 cells by electroporation (FIG. 2B). Subsequently the recombinase catalyses the intermolecular recombination between attP* and chromosomal attH (hatched rectangle marked with att “H/HOH′”) leading to the integration of the vector pEL13 into the genome of the BL60 cells. The cells which stably incorporated the vector may be selected and identified by a PCR with the primer pair attX1/B2 (arrows marked with “attX1” and “B2”). EcoRV and SphI designate the restriction enzyme recognition sites of the respective restriction enzymes.

FIG. 9 shows schematically the detection of the intermolecular recombination between attP* (pEL13) and attH in BL60 cells. Genomic DNA was isolated and amplified with the primer pair attX1/B2 (FIG. 8) from 31 different cell populations after electroporation of pEL13 and a following selection over several weeks. The PCR products were separated and made visible by agarose gel electroporesis. The position of the expected product (295 bp) is marked in the gels by an arrow. Subsequently the products were analyzed further by DNA sequencing. On the right margin a DNA ladder is located.

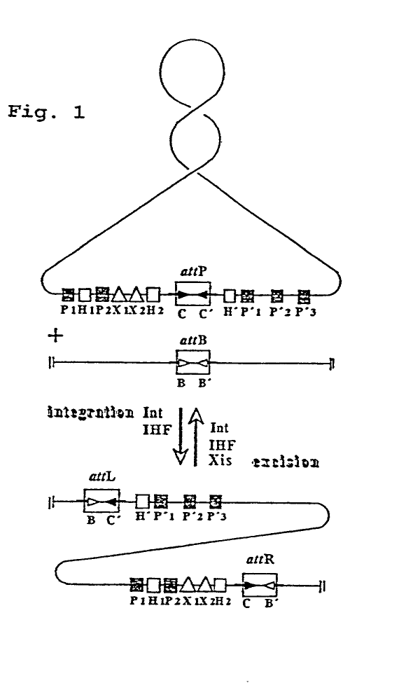

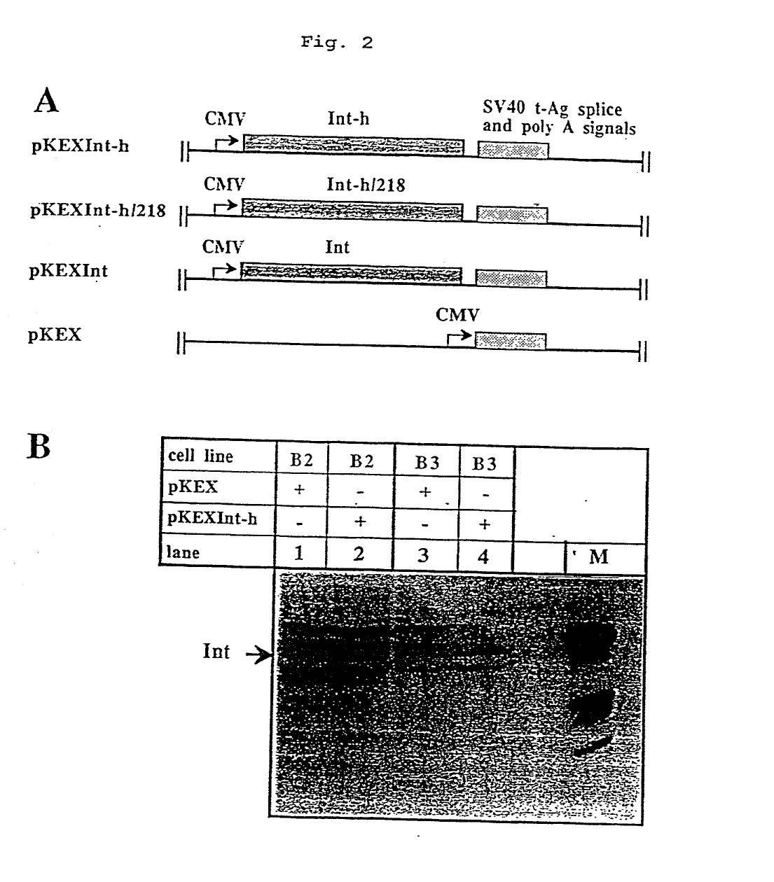

### DETAILED DESCRIPTION OF THE INVENTION

### Examples

1. Production of Expression and Substrate Vectors

1.1 Expression Vectors

The eukaryotic expression vectors for wild-type Int (pKEXInt), Int-h (pKEXInt-h), Int-h/218 (pKEXInt-h/218) and pEL13 are derivatives of pKEX-2-XR (Rittner et al. (1991), Methods Mol. Cell. Biol., 2, pp. 176). Said vector includes the human cytomegalo virus promotor/enhancer element (CMV) and the RNA splicing and polyadenylation signal elements of the small simian virus 40 (SV40) tumor antigen. The Int genes were cloned by PCR with the following primers: (3343) 5′-GCTCTAGACCACCATGGGAAGAAGGCGAAGTCA-3′, located at the 5′ end of the Int gene and (3289) 5′-AAGGAAAGCGGCCGCTCATTATTTGATTTCAATTTTGTCC-3′, located at the 3′ end.

The amplification was carried out after a first denaturation step at 95° C. (4 min.) with 30 cycles of denaturation (95° C., 45 sec.), primer binding (55° C., 45 sec.), DNA synthesis (72° C., 2 min.) and a final synthesis step for 4 min at 72° C. The resulting PCR fragment was cloned into the pKEX-2-XR vector with XbaI and NotI. Int-h was generated from the vector pHN16 as a template (Lange-Gustafson, B. and Nash, H. (1989) J. Biol. Chem., 259, pp. 12724). Wild-type Int and Int-h/218 were generated from pTrcInt and pTrcInt-h/218 as template, respectively; (Christ, N. and Dröge, P. (1999) J. Mol. Biol., 288, pp. 825). pEL13 carries in addition to the Int-h gene a copy of attP*.

Starting from attP attP* was constructed by PCR mutagenesis. The following oligonucleotides were used:

(O3)
5′-GTTCAGCTTTTTGATACTAAGTTG-3′,
(O4)
5′-CAACTTAGTATCAAAAAGCTGAAC-3′,
(PC)
5′-TTGATAGCTCTTCCGCTTTCTGTTACAGGTCACTAATACC-3′ and
(PD)
5′-ACGGTTGCTCTTCCAGCCAGGGAGTGGGACAAAATTGA-3′.

The amplification was carried out after a first denaturation step at 95° C. (4 min.) with 30 cycles of denaturation (95° C., 45 sec.), primer binding (57° C., 1 min. 30 sec.), DNA synthesis (72° C., 1 min. 30 sec.) and a final synthesis step for 4 min at 72° C. The PCR product was incubated with the restriction enzyme SapI and ligated with pKEXInt-h cleaved with SapI. The control plasmid pKEX carries no Int gene.

1.2 Substrate Vectors

The substrate vectors are derivatives of pEGFP (Clontech). The recombination cassettes are under the control of the CMV promoter, guaranteeing a strong constitutive expression. pGFPattB/attP was constructed by cutting the GFP gene (green fluorescence protein) out of pEGFP by AgeI and BamHI first. The wild-type attB sequence was inserted as double stranded oligonucleotide into the vector cleaved with AgeI using the following oligonucleotides:

(B1OB)
5′-CCGGTTGAAGCCTGCTTTTTTATACTAACTTGAGCGAACGC-3 and
(BOB1)
5′-AATTGCGTTCGCTCAAGTTAGTATAAAAAAGCAGGCTTCAA-3′.

The wild-type attP sequence was amplified by PCR from the vector pAB3 (Dröge, P. and Cozzarelli, N. (1989) Proc. Natl. Acad. Sci., 86, pp. 6062) using the following primers:

| | |
|---|---|
| (p7) | 5′-TCCCCCCGGGAGGGAGTGGGACAAAATTGA-3′ and |
| (p6) | 5′-GGGGATCCTCTGTTACAGGTCACTAATAC-3′. |

The amplification was carried out after a first denaturation step at 95° C. (4 min.) with 30 cycles of denaturation (95° C., 45 sec.), primer binding (54° C., 30 sec.), DNA synthesis (72° C., 30 sec.) and a final synthesis step for 4 min at 72° C. The PCR fragment carrying attP was digested with XmaI and BamHI and ligated with a restriction fragment carrying the GFP gene. Said GFP restriction fragment was generated from pEGFP with AgeI and EcoRI. The ligation product was cloned into the attB carrying vector cleaved with MfeI/BamHI. The resulting substrate vector carries the GFP gene in inverted orientation with regard to the CMV promotor whose functionality in integrative recombinations was tested with wild-type Int in E. coli.

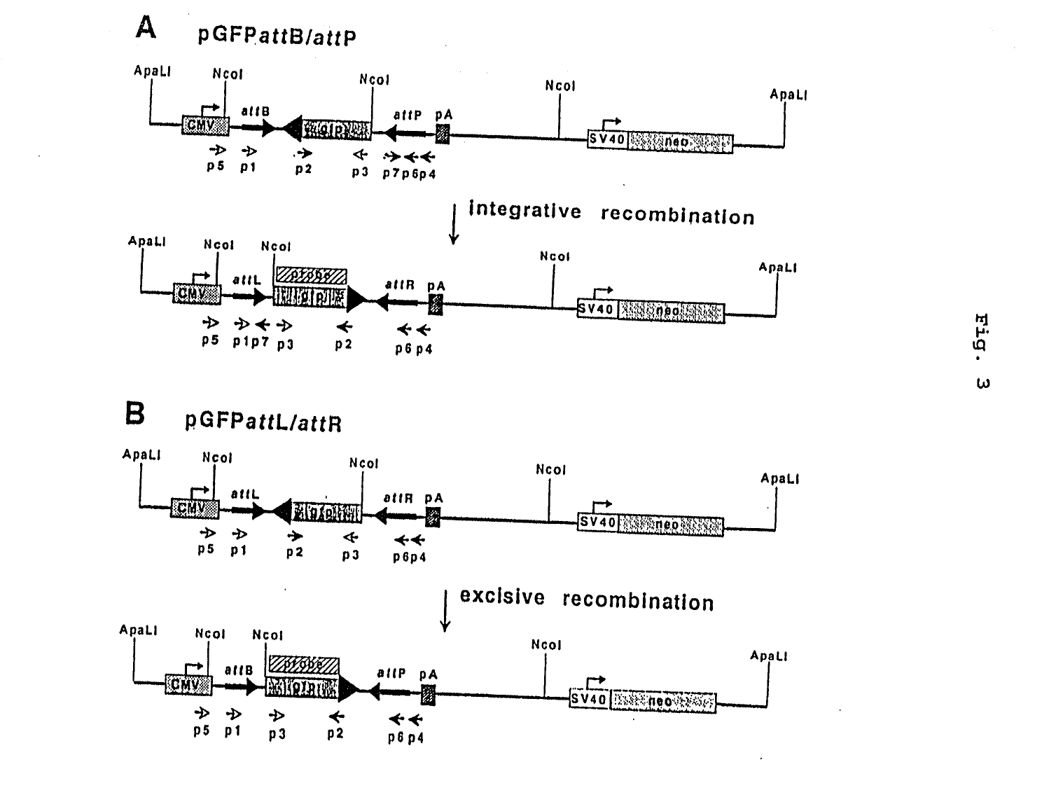

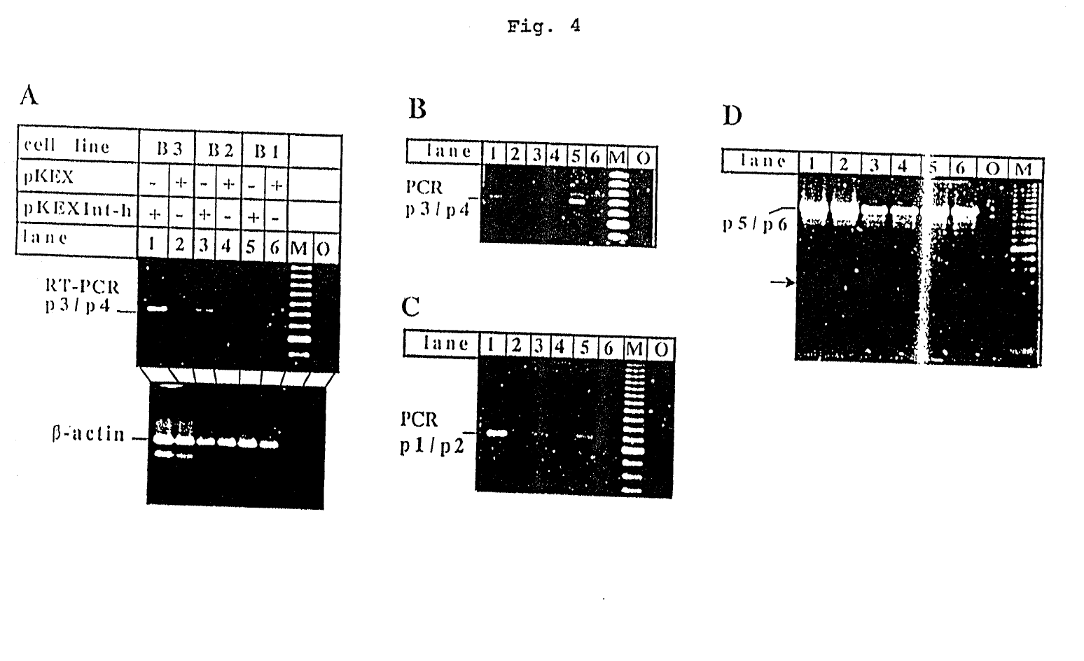

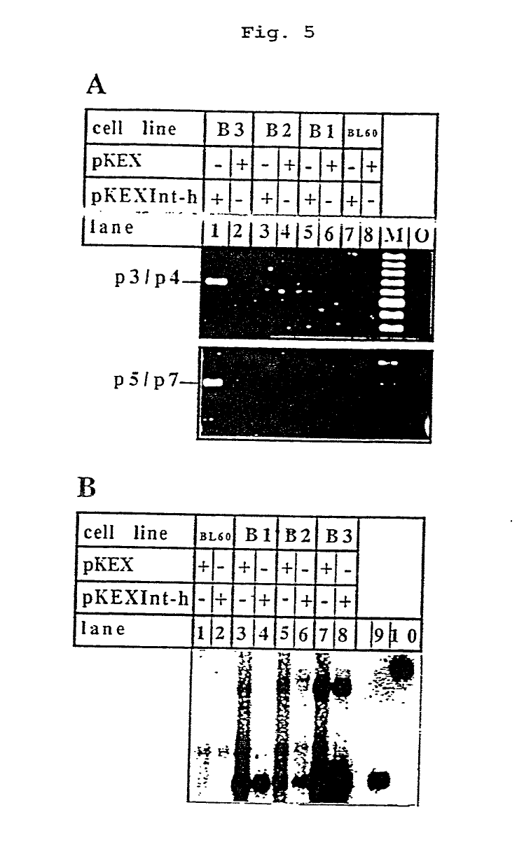

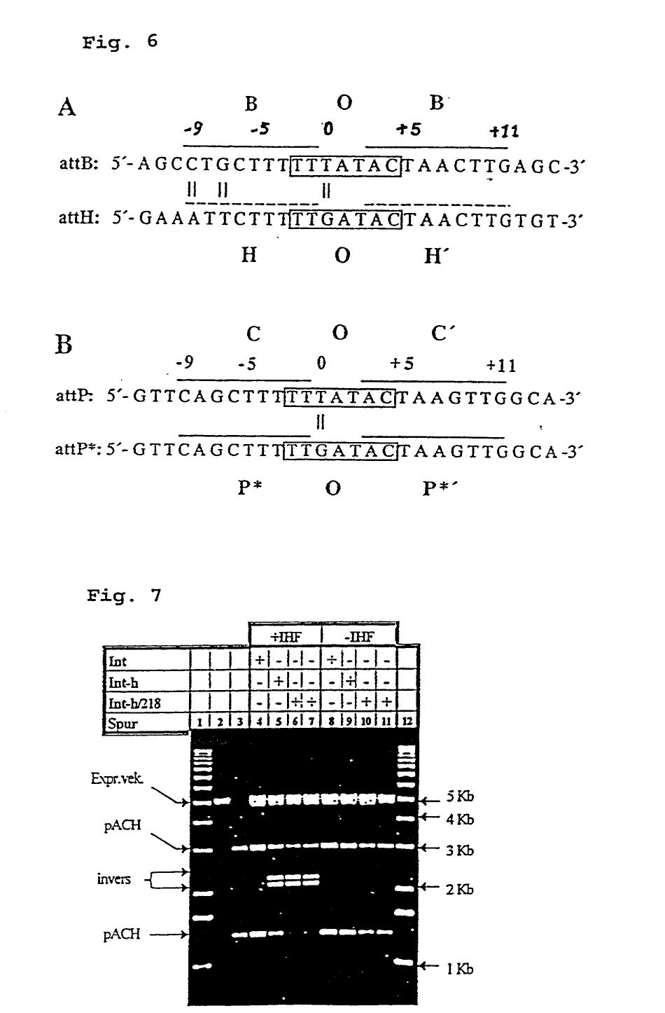

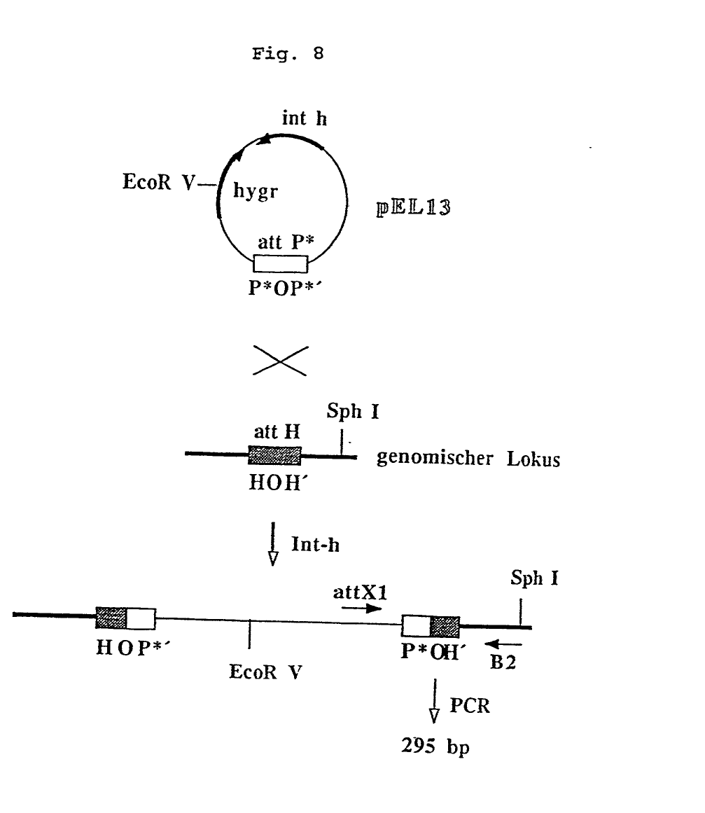

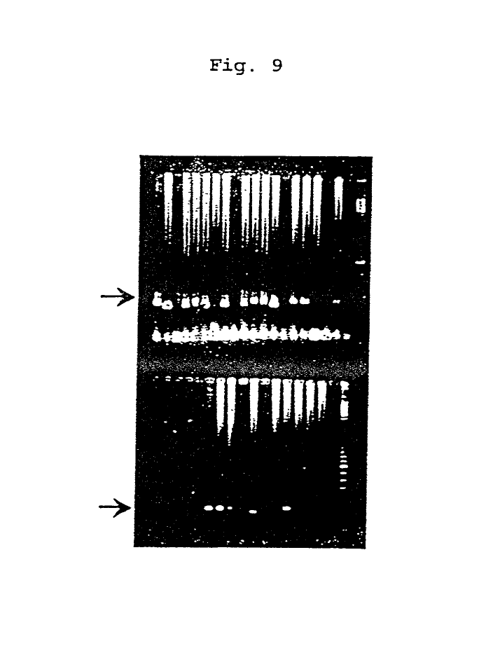

With the exception of the recombination sequences, pGFPattL/attR is identical to pGFPattB/attP. The vector was constructed by first recombining pGFPattB/attP in E. coli leading to the formation of attL and attR. The subsequently with regard to the CMV promotor correctly orientated GFP gene was excised with a partial restriction reaction with BsiEI and HindIII. The GFP gene was first of all amplified by PCR using the following primers to insert it in inverted orientation with regard to the CMV promotor:

| | |
|---|---|
| (p2) | 5′-AATCCGCGGTCGGAGCTCGAGATCTGAGTCC-3′ |
| | and |
| (p3) | 5′-AATCCCAAGCTTCCACCATGGTGAGCAAGGG-3′ |
| | (FIG. 3). |

The amplification was carried out after a first denaturation step at 95° C. (4 min.) with 30 cycles of denaturation (95° C., 45 sec.), primer binding (56° C., 45 sec.), DNA synthesis (72° C., 1 min.) and a final synthesis step for 4 min at 72° C. The PCR fragment was cleaved with HindIII and BsiEI subsequently and integrated into the partially cleaved vector including attL and attR in inverted orientation. Thus, pGFPattL/attR shows the same global structure as pGFPattB/attP with the exception of the presence of attL/attR instead of attB/attP.

The human attB homologue, attH, was amplified from purified human DNA by PCR using the following primers:

| | |
|---|---|
| (B3) | 5′-GCTCTAGATTAGCAGAAATTCTTTTTG-3′ and |
| (B2) | 5′-AACTGCAGTAAAAAGCATGCTCATCACCCC-3′. |

The amplification was carried out after a first denaturation step at 95° C. (5 min.) with 30 cycles of denaturation (95° C., 45 sec.), primer binding (42° C., 1.45 min.), DNA synthesis (72° C., 1.45 min.) and a final synthesis step for 10 min at 72° C. The primer sequences for the generation of attH have been taken from an EST (Accession No.: N31218; EMBL-Database). The uncompleted sequence of attH as present in the database was verified and completed by sequencing of the isolated PCR product (192 bp). Subsequently, the fragment was digested with XbaI and PstI and inserted into the correspondingly treated vector pACYC187 (New England Biolabs). AttP* was generated by targeted mutagenesis as described (Christ, N. and Dröge, P. (1999) J. Mol. Biol., 288, pp.825) and inserted into the attH carrying vector in inverted orientation to attH. This construction leads to the test vector pACH.

Plasmid DNAs were isolated from E. coli strain DH5α (Hanahan, D. (1983) J. Mol. Biol., 166, pp.557) by affinity chromatography (Qiagen, Germany). Expression and substrate vectors as well as all PCR generated constructs were controlled by means of the fluorescent based 373A DNA-Sequencing system (Applied Biosystems). PCR reactions were carried out by the “Master Mix Kit” (Qiagen, Germany) and the resulting products were analyzed by an agarose gel electrophoresis (0.8% w/v) in TBE buffer.

2. Cell Culture and the Construction of the Reporter Cell Lines

The transient expression and recombination analyses were carried out with a human Burkitt's Lymphoma cell line (BL60; (Wolf, J. et al., (1990) Cancer Res., 50, pp. 3095)). BL60 cells were cultured in RPMI1640 medium (Life Technologies, Inc.) enriched with 10% fetal calf serum and including 2 mM L-glutamine, streptomycin (0.1 mg/ml) and penicillin (100 units/ml).

BL60 reporter cell lines with either pGFPattB/attP or pGFPattL/attR stably integrated into the genome were constructed as follows: about 20 μg of each vector were linearized with ApaLI purified with phenol/chloroform extractions, precipitated with ethanol and introduced into about 2×10⁷ cells by electroporation at 260 V and 960 mF using the “Bio-Rad Gene Pulser”. Stable cell lines were selected with G418/Genetizin (300 μg/ml) and characterized subsequently by PCR, DNA sequencing and Southern analysis.

3. In vivo Recombination Analyses

To perform intramolecular recombination in vivo about 2×10⁷ cells of the respective BL60 reporter cell line was transfected with 40 μg of each circular expression vector by electroporation as described in example 2. The cells were harvested after 72 hours by centrifugation and the genomic DNA of half of the cells was isolated by affinity chromatography according to the instructions of the manufacturer (Qiaamp Blood Kit, Qiagen, Germany). From half of the cells either RNA was isolated (Rneasy kit, Qiagen, Germany) or a cell lysate was prepared for the Western analysis (see example 4).

The recombination analyses with pACH were carried out in E. coli as described above (Christ, N. and Dröge, P. (1999) J. Mol. Biol., 288, pp.825) using the recombinases Int, Int-h and Int-h/218. The expected recombination of pACH leads to an inversion and was proved by restriction analysis with HindIII and AvaI.

Intermolecular recombination for an integration of pEL13 into the genomic localized attH locus of BL60 cells was carried out as follows: 2×10⁷ cells were transfected with 20 μg circularized pEL13 via electroporation as described above. The cells were plated in a concentration of 1×10⁶ cells/ml selection medium (200 μg/ml hygromycin B) after 48 hours and incubated for 6 to 8 weeks. From a portion of the respective surviving cell populations genomic DNA was prepared after the incubation according to the instructions of the manufacturer (Qiaamp Blood Kit, Qiagen, Germany).

To prove intramolecular, integrative and excisive recombination 0.4 μg genomic DNA was amplified by PCR using 20 to 50 pmol of the following primers:

| | |
|---|---|
| (p1) | 5′-GGCAAACCGGTTGAAGCCTGCTTTT-3′; |
| (p2) | 5′-AATCCGCGGTCGGAGCTCGAGATCTGAGTCC-3′; |
| (p3) | 5′-AATCCCAAGCTTCCACCATGGTGAGCAAGGG-3′; |
| (p4) | 5′-AACCTCTACAAATGTGGTATGG-3′, |
| (p5) | 5′-TACCATGGTGATGCGGTTTTG-3′; |
| (p6) | 5′-GGGGATCCTCTGTTACAGGTCACTAATAC; |
| (p7) | 5′-TCCCCCCGGGAGGGAGTGGGACAAAATTGA-3′. |

The amplification was carried out after a first denaturation step at 95° C. (5 min.) with 30 cycles of denaturation (95° C., 45 sec.), primer binding (57° C., 45 sec.), DNA synthesis (72° C., 1.5 min.) and a final synthesis step for 4 min at 72° C.

Intermolecular integrative recombination of pEL13 was detected as follows. About 400 ng of the genomic DNA of surviving cell populations was incubated with the following oligonucleotides as PCR primers:

| | |
|---|---|
| (attx1) | 5′-AGTAGGAATTCAGTTGATTCATAGTGACTGC-3′ and |
| (B2) | 5′-AACTGCAGTAAAAAGCATGCTCATCACCCC-3′. |

The amplification was carried out after a first denaturation step at 95° C. (4 min.) with 30 cycles of denaturation (95° C., 45 sec.), primer binding (52° C., 45 sec.), DNA synthesis (72° C., 45 sec.) and a final synthesis step for 4 min at 72° C.

The reverse transcriptase PCR (RT-PCR) was carried out with 4 μg isolated RNA. First, the cDNAs were synthesized using oligo-dT primers according to the instructions of the manufacturer (First Strand Synthesis Kit, Pharmacia). Second, a quarter of said cDNAs was used as a template for the subsequent PCR using primers p3 and p4. To test for deletion instead of inversion isolated genomic DNA was amplified with the primers p5 and p6. Beta actin transcripts were analyzed starting from said cDNAs using the primers

| | |
|---|---|
| (AS) | 5′-TAAAACGCAGCTCAGTAACAGTCCG-3′ and |
| (S) | 5′-TGGAATCCTGTGGCATCCATGAAAC-3′. |

The PCR conditions were the same as described for p1 to p7.

Southern analyses were essentially carried out according to the protocol of Sambrook, J. (1989) Molecular Cloning (2 nd Edt.) Cold Spring Harbor Laboratory Press. About 10 μg of genomic DNA was fragmented with NcoI, separated by agarose gel electrophoresis (0.8% w/v) in TBE buffer and transferred to a nylon membrane over night. The GFP probe for the detection of the recombination was generated by PCR using the primers p2 and p3. The radioactive labeling was carried out using ³²P labeled dATP and dCTP according to the instructions of the manufacturer (Megaprime, Amersham).

4. Western Analysis

Cell lysates of transiently transfected cells were generated by boiling the cells in probe buffer (New England Biolabs) for 5 min. The proteins were separated in a 12.5% SDS polyacrylamid gel according to their molecular weight and transferred onto a nitrocellulose membrane (Immobilon P, Millipore) over night. The membrane was treated with 1% blocking solution (BM Chemiluminescence Western Blotting Kit, Boehringer Mannheim, Germany) and incubated with murine polyclonal antibodies directed against wild-type Int at a dilution of 1:50.000 (antibodies from A. Landy, USA). The secondary antibodies coupled to peroxidase were used to visualize the location of the integrase in the gel (BM Chemiluminescence Western Blotting Kit; Boehringer Mannheim, Deutschland). E. coli cell extracts containing wild-type Int were used as a control.

5. Results

5.1 Sythesis of Int-h in BL60 Cells

To test whether Int-h can catalyze recombination in human cells it was necessary to demonstrate that the recombinase can be synthesized from said cells. Therefore, the eukaryotic expression vector, pKEXInt-h, carrying the Int-h gene under the control of the CMV promotor was integrated. After the introduction of pKEXInt-h into two different BL60 reporter cell lines, namely B2 and B3, complete and correctly modified transcripts being specific for the Int-h gene could be detected by RT-PCR analysis. Cell lysates were investigated in a Western analysis 72 hours after electroporation with pKEXInt-h. The detection of the recombinase was carried out with murine polyclonal antibodies directed against wild-type Int. pKEX was introduced into the cells as a control.

The results demonstrate that a protein having the expected molecular weight was present in the cells treated with pKEXInt-h in the electroporation. Said protein was not detectable if the control vector pKEX was used.

5.2 Int-h Catalyzed Integrative Intramolecular Recombination in Human Cells

The Western analysis demonstrated that the Int-h protein is synthesized form the two reporter cell lines starting from the vector pKEXInt-h. Said cells contain a substrate vector, pGFPattB/attP, as a foreign DNA stably integrated into their genome. The two recombination sequences for the integrative recombination, namely attB and attP, are located in inverted orientation to each other and flank the gene for GFP. The GFP gene itself is located in inverted orientation to the CMV promotor which is located upstream of attB. Recombination between attB and attP by the Int-h leads to the inversion of the GFP gene and, thus, to its expression. Three reporter cell lines (B1 to B3) were constructed in total. Southern analysis of their genomic DNA demonstrated that several copies of pGFPattB/attP as direct repeats have been integrated into the genome of B1 and B3, whereas the cell line B2 contains only one copy. The integrated sequences were verified by PCR and subsequent sequencing.

To test for the recombination between attB and attP pKEXInt-h and pKEX were introduced seperatly into the cell lines. The cells were harvested 72 hours after electroporation, RNA was isolated from a portion of said cells and investigated for GFP expression by RT-PCR using the primer pair p3/p4. Said primers amplified a 0.99 kb long DNA fragment only, if the GFP gene was inverted due to recombination. The results demonstrated that the product was detectable in all three cell lines. If pKEX was introduced into the cells no product was detectable. DNA sequence analyses of the isolated PCR products confirmed that the coding region of the GFP gene was transcribed and that attR instead of attP was detectable in the transcript. As control of the RNA amount in all six cell preparations as well as for the successful first strand DNA synthesis by the reverse transcriptase the endogenous β-actin transcript was analyzed by PCR. The results demonstrated that the transcript was present in almost the same amounts.

Recombination was detected also by direct PCR of genomic DNA. The results demonstrated that the expected products could only be detected using the primer pairs p3/p4 (0.99 kb) and p1/p2 (0.92 kb) if pKEXInt-h was introduced into the cells. The analysis of said products by DNA sequencing confirmed that attR and attL were present in the genome and that the GFP gene was inverted by the recombination. These experiments have been repeated three times wherein the recombination between attB and attP was detectable in all three cell lines by RT-PCR and/or PCR. A detection of the deletion of the GFP gene by PCR was negative with the primer pair p5/p6. Only the expected 1.3 kb fragment resulting from the integrated vector could be amplified.

The strongest signal showing an inversion between attB and attP in the PCR was repeatedly obtained with the cell line B3 in a further experiment. As a result genomic DNA was fragmented by NcoI and examined by a Southern analysis by means of a GFP gene as a probe. The results demonstrated that the restriction fragment of genomic DNA was detectable in the cell line B3 which was expected as a result of the Inversion between attB and attP.

To test whether wild-type Int and the mutant Int-h/218 could catalyze intramolecular integrative recombination also the vectors pKEXInt-h, pKEXInt-h/218, pKEXInt and as a control pKEX were introduced into the reporter cell line B3 in a further experiment by electroporation as described in example 2, infra. Genomic DNA was isolated after 72 hours and tested for recombination via PCR with the primer pairs p5/p7 and p3/p4 as described in example 3, infra. The results demonstrated that both Int mutants could catalyze recombination between attB and attP, however, the wild-type Int was inactive.

5.3 Excisive Recombination between attL and attR was not Detectable

Because Int-h could catalyze also excisive recombination between attL and attR in the absence of the co-factors IHF and Xis three BL60 reporter cell lines were constructed having stably integrated the vector pGFPattL/attR into the genome. Again, said cell lines included the GFP gene in inverted orientation with regard to the CMV promoter, however, flanked by attL and attR instead of attB and attP. The recombination analyses were carried out with pKEXInt-h as expression vector for the recombinase as described in example 3, infra, however, they demonstrated that neither inversion nor deletion was detectable between attL and attR by means of RT-PCR or PCR.

5.4 Identification and Characterization of a Naturally Occurring Nucleotide Sequence in the Human Genome Similar to AttB

Both Int recombinase mutants catalyze integrative intramolecular recombination in human cells as demonstrated in example 3. One of the two recombination sequences involved in this reaction, namely attB, is 21 bp long and a natural part of the E. coli genome. It could be demonstrated that some differences in the sequence of the so-called core recognition region of attB are tolerated by Int-h in a recombination with attP (Nash (1981) Annu. Rev. Genet., 15, pp143). The presence of a functional sequence homologous to attB in the human genome is possible from a statistically point of view. The inventors could identify a still incomplete sequence as part of an expressed sequence tag (EST) in a database search. Said sequence was then isolated by PCR from human DNA and cloned. A DNA sequence analysis completed the sequence and a further Southern analysis with genomic DNA of the BL60 cells demonstrated that said sequence is a part of a still unknown human gene present in the genome as a single copy gene.

Said sequence, herein designated as attH, differs from the wid-type attB sequence at three positions. Two of the nucleotides are located in the left (B) Int core recognition region and the third is part of the so-called overlap region. Because the identity of the overlap region of the two recombination sequences is a prerequisite for an efficient recombination by Int-h the respective nucleotide at position 0 in the overlap of attP was changed from thymidin to guanine leading to attP*. AttH and attP* were incorporated as inverted sequences in a vector (pACH) and tested for recombination in E. coli. The results demonstrated that Int-h and Int-h/218 catalyzed inversion between attH and attP* in the absence of IHF. DNA sequence analyses of the isolated recombination products confirmed that recombination between attH and attP* occurred with the expected mechanism. By contrast, wild-type Int can recombine attH/attP* even in the presence of IHF only very inefficiently. Thus, attH is a potential integration sequence for Int-h catalyzed integration of foreign DNA including a copy of attP*.

5.5 Integrative Intermolecular Recombination between attH and attP* in Human Cells

pEL13 was constructed to demonstrate whether attH as a natural part of the human genome can recombine with attP* in an intermolecular reaction. Said vector includes a copy of attP* besides the Int-h gene under the control of the CMV promotor and the resistance gene hygromycin as a selection marker. After introduction of pEL13 into BL60 cells Int-h could be synthesized and catalyzed the intermolecular recombination between genomic attH and attP* as part of pEL13.

PEL13 was introduced into BL60 cells by means of electroporation as described in example 2. Said cells were put under selection pressure and diluted after 72 hours. Surviving cell populations were examined for recombination events after 6 to 8 weeks by PCR with the primer pair attx1/B2. The results demonstrated that in 13 of the 31 surviving cell populations an integration in attH was detectable. DNA sequence analyses of the PCR products from different approaches confirmed their identity as recombination products.

We claim:

1. A method of sequence specific recombination of DNA in a eukaryotic cell, comprising
a) introducing a first DNA sequence into a cell,
b) introducing a second DNA sequence into a cell, and
c) performing the sequence specific recombination by a bacteriophage lambda integrase Int.

2. Method of sequence specific recombination of DNA in a eukaryotic cell having a first DNA sequence in its genome, either naturally occurring or being introduced previously by DNA recombination, comprising the steps b) and c) defined in claim 1.

3. Method according to claim 1 or 2, wherein said first DNA sequence comprises an attB sequence according to SEQ ID NO:1 or a derivative thereof and said second DNA sequence comprises an attP sequence according to SEQ ID NO:2 or a derivative thereof.

4. Method according to claim 1 or 2, wherein said first DNA sequence comprises an attL sequence according to SEQ ID NO:3 or a derivative thereof and said second DNA sequence comprises an attR sequence according to SEQ ID NO:4 or a derivative thereof, wherein in step c) additionally a Xis factor is present.

5. Method according to anyone of claims 1 to 4, wherein additionally a third or a third and fourth DNA sequence comprising an Int gene or an Int gene and a Xis factor gene, respectively, is introduced into the cell.

6. Method according to claim 5, said third or said third and/or fourth DNA sequence further comprising a regulatory DNA sequence effecting a spatial and/or temporal expression of the Int gene and/or the Xis factor gene.

7. Method according to anyone of claims 1 to 6, wherein said Int is a modified integrase.

8. Method according to claim 7, wherein said modified Int is Int-h or Int-h/218.

9. Method according to anyone of claims 1 to 8, wherein in step c) additionally an “integration host factor” (IHF) is involved.

10. Method according to anyone of claims 1 to 9, said first and/or second DNA sequence further comprising DNA sequences effecting an integration of said first and/or second DNA sequence into the genome of the eukaryotic cells by homologous recombination.

11. Method according to anyone of claims 1 to 10, said first and/or second DNA sequence further comprising a nucleic acid sequence coding for a polypeptide of interest.

12. Method according to claim 11, wherein said polypeptide of interest is a structural protein, an endogenous or exogenous enzyme, a regulatory protein or a marker protein.

13. Method according to anyone of claims 1 and 3 to 12, wherein said first and second DNA sequence are introduced into the eukaryotic cell on the same DNA molecule.

14. Method according to anyone of claims 1 to 13, wherein said eukaryotic cell is a mammalian cell.

15. Method according to claim 14, wherein said mammalian cell is a human, simian, mouse, rat, rabbit, hamster, goat, bovine, sheep or pig cell.

16. Method according to anyone of claims 1 to 3 and 5 to 15, further comprising
d) performing after a first sequence specific recombination of DNA according to the steps a) to c) or a) and b) without a Xis factor a second sequence specific recombination of DNA by an Int and a Xis factor.

17. Method according to claim 16, further introducing a further DNA sequence into said cells, the further DNA sequence comprising a Xis factor gene.

18. Method according to claim 17, wherein said further DNA sequence comprises further a regulatory DNA sequence effecting a spatial and/or temporal expression of said Xis factor gene.

19. The use of an attB sequence according to SEQ ID NO:1 or a derivative thereof and an attP sequence according to SEQ ID NO:2 or a derivative thereof, or an attL sequence according to SEQ ID NO:3 or a derivative thereof and an attR sequence according to SEQ ID NO:4 or a derivative thereof in a sequence specific recombination of DNA in eukaryotic cells.

20. Nucleic acid sequence according to SEQ ID NO:5 or a derivative thereof.

21. Vector, comprising a nucleic acid sequence according to SEQ ID NO:5 or a derivative thereof and a further nucleic acid sequence coding for a therapeutic gene or a DNA fragment thereof.

22. Vector according to claim 21, wherein said therapeutic gene is the CFTR gene, ADA gene, LDL receptor gene, β globin gene, Factor VIII gene or Factor IX gene, alpha-1-antitrypsin gene or the dystropin gene or a gene fragment of one of said genes.

23. Vector according to claim 21 or 22, wherein said further nucleic acid sequence comprises further expression and/or transcription elements.

24. Vector according to anyone of claims 21 to 23 for the use as a medicament in the human or veterinary medicine.

25. Use of a vector according to anyone of claims 21 to 23 for the manufacture of a medicament for the somatic gene therapy.

26. Eukaryotic cell, obtainable by subjecting said eukaryotic cell of claim 1 or 2 to the method according to anyone of claims 1 to 18.

27. Transgenic organism comprising at least one cell according to claim 26.

28. The organism according to claim 27, wherein said organism is a mouse, rat rabbit or hamster.
